(19) United States
(12) Patent Application Publication
(10) Pub. No.: US 2005/0027234 A1
(43) Pub. Date: Feb. 3, 2005

(54) Surgical implant and method of accessing cerebrospinal fluid

(76) Inventors: Donna Jean Waggoner, Kalamazoo, MI (US); Allen Edwin Buhl, Kalamazoo, MI (US); Steven Michael Kuhlman, Richland, MI (US); Matthew Crosby Flegal, Kalamazoo, MI (US)

Correspondence Address: FLYNN, THIEL, BOUTELL & TANIS, P.C., 2026 RAMBLING ROAD, KALAMAZOO, MI, 49008-1699, US

(21) Appl. No.: 10/870,478

(22) Filed: Jun. 17, 2004

Related U.S. Application Data
(60) Provisional application No. 60/479,402, filed on Jun. 18, 2003.

(51) Int. Cl. A61M 5/00

(52) U.S. Cl. 604/8

(57) ABSTRACT

A surgical implant and method of gaining access to cerebrospinal fluid in the brain. The implant includes an upper housing part which is positioned subcutaneously on the skull and a lower cage-like member which depends downwardly from the housing part. The housing part defines a chamber or reservoir therein which communicates with openings defined in the lower member. The implant is embedded between portions of the brain so that the lower member projects between the cerebellum and the cerebrum and holds same apart in order to access cerebrospinal fluid which pools in this area. The cerebrospinal fluid is accessed for sampling or dosing purposes through a septum provided within the upper housing part.

### FIELD OF THE INVENTION

This invention generally relates to an implant and method of gaining access to cerebrospinal fluid (csf) in the brain.

### BACKGROUND OF THE INVENTION

It is often necessary for research and treatment purposes to have access to cerebrospinal fluid from conscious patients. For example; by sampling or collecting cerebrospinal fluid the progression of various brain diseases, infections, or other ailments can be monitored on a regular basis. In research, cerebrospinal fluid sampling is often required to monitor drug levels as well as to monitor changes in physiological parameters in the cerebrospinal fluid. Further, it is often desirable or necessary to administer therapeutic agents directly into the cerebrospinal fluid to bypass the blood-brain barrier.

Various devices and methods have been developed for the purpose of accessing cerebrospinal fluid in animals. One such device is a guide cannula which is implanted within the skull of the animal. One or more of these guide cannulas are secured into the skull of the animal and extend to touch the surface of the dura mater on the surface of the brain so that each of the guides is aligned (but not in contact) with one of the lateral ventricles of the brain. The guides are implanted for the purpose of permitting repeated sampling of cerebrospinal fluid over a predetermined span of time, and thus the guides are left within the skull of the animal and are accessed via a collection needle placed through the skin and muscle located above the respective guides following a surgical-style preparation of the skin over the guides. The needle is inserted into the guide cannula and is guided thereby into the corresponding lateral ventricle to collect cerebrospinal fluid. One of the disadvantages of this arrangement is that the guide cannula locks to the skull of the animal with screw-threads, which can cause difficulty with respect to successfully aligning the needle guide in relation to the lateral ventricle. Further, the screw-threads often result in improper placement of the guide cannula when the sloped surface of the skull catches the threads and pulls the implant out of proper alignment. Another disadvantage of the above arrangement is that same is typically not suitable for use on small animals due to the extremely small size of the lateral ventricles and thinness of the skull.

The present invention is directed to an implant for accessing cerebrospinal fluid from the brain, which implant includes an upper housing which is fixed to the skull and a lower cage-like member which protrudes from the lower side of the housing through a hole in the skull and dura mater and is positioned within a space, called the transverse fissure, defined in the brain between the cerebrum and the cerebellum where pools of cerebrospinal fluid are located. The housing defines a reservoir therein in communication with a hollow interior of the cage-like member. Since cerebrospinal fluid is under pressure within the brain, this fluid flows into the cage-like member and up into the reservoir defined in the housing. The upper part of the housing is closed off with a septum, and cerebrospinal fluid is accessed and withdrawn with a collection needle which is used to penetrate the septum through the skin to collect fluid from the reservoir. Alternatively, therapeutic agents can be dosed directly into the cerebrospinal fluid with a dosing needle which penetrates the septum and delivers the drug into the reservoir for circulation. The implant according to the invention thus serves to create a closed system over a surgically-created opening in the skull and dura mater so as to create a reservoir or access port for cerebrospinal fluid from around the brain.

Other objects and purpose of the invention will be apparent to persons familiar with devices of this type upon reading the following description and inspecting the accompanying drawings.

### BRIEF DESCRIPTION OF THE DRAWINGS

FIG. 1 is front elevational view of the implant according to the invention.

FIG. 2 is a plan view of the implant.

FIG. 3 is a cross-sectional view of the implant taken generally along line 3-3 in FIG. 1.

FIG. 4 is a cross-sectional view of the implant taken generally along line 4-4 in FIG. 3.

FIG. 5 is a plan view of the brain of an animal.

FIG. 6 is a cross-sectional view taken generally along line 6-6 in FIG. 5.

FIG. 7 is an enlarged, cross-sectional view of the implant in position in the brain of the animal.

Certain terminology will be used in the following description for convenience in reference only, and will not be limiting. For example, the words “upwardly”, “downwardly”, “rightwardly” and “leftwardly” will refer to directions in the drawings to which reference is made. The words “front” and “rear” will be used to refer to the spatial orientation of components of the implant in relation to the anatomical front and rear of the animal, respectively. The words “inwardly” and “outwardly” will refer to directions toward and away from, respectively, the geometric center of the arrangement and designated parts thereof. Said terminology will include the words specifically mentioned, derivatives thereof, and words of similar import.

### DETAILED DESCRIPTION

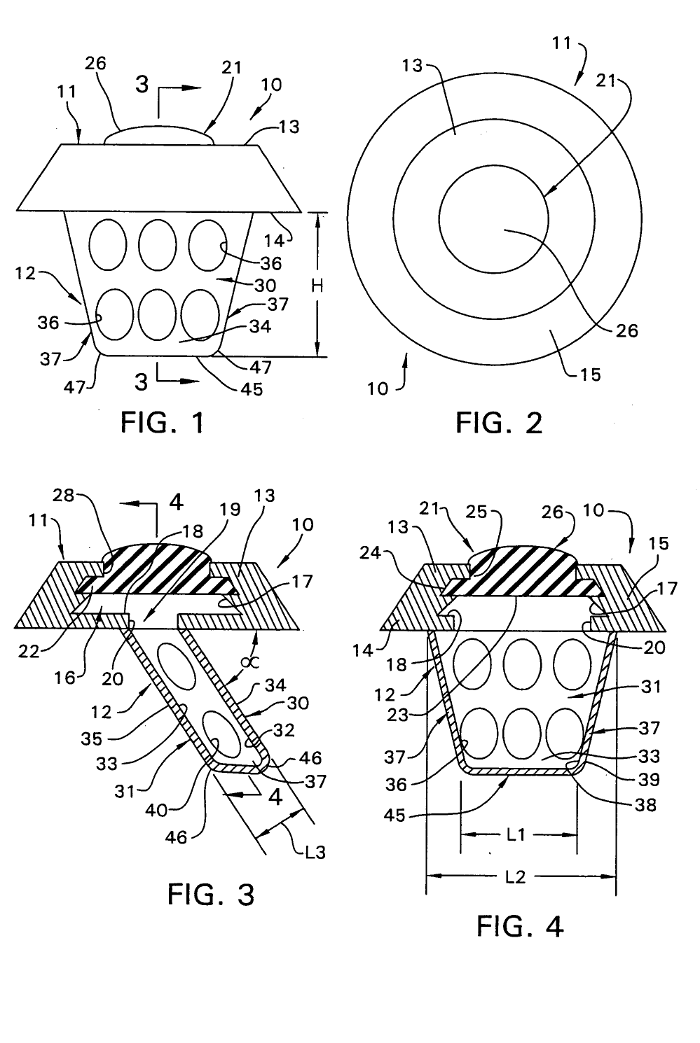

Referring to FIGS. 1-4, an implant 10 is illustrated according to the present invention. The implant 10 generally includes a rigid upper housing part 11 and a generally open, rigid and cage-like lower member 12 which is cantilevered downwardly from housing part 11.

Upper housing part 11 defines a generally flat and annular upper wall 13 which is vertically spaced from and generally parallel with a generally flat and annular lower wall 14. An annular side wall 15 extends between and adjoins upper and lower walls 13 and 14. Side wall 15 angles outwardly as same projects downwardly from upper wall 13 towards lower wall 14, and has a frusto-conical shape when viewed from the side. Housing part 11 defines a chamber or reservoir 16 therein. Reservoir 16 is defined by an inner surface 17 of side wall 15 which is inclined and generally parallel to an outer surface of side wall 15, and a generally horizontally oriented upper surface 18 of lower wall 14 which is spaced upwardly from and generally parallel to a lower surface of lower wall 14. Housing part 11 additionally defines therein a passage 19 which communicates with reservoir 16, projects downwardly from surface 18 through lower wall 14, and opens through the lower surface of lower wall 14. In the illustrated embodiment, passage 19 is defined by an inner upright surface 20 of lower wall 14 which is perpendicular to upper surface 18.

A septum 21 is mounted or embedded within an upwardly opening recess 28 defined in upper housing part 11. Septum 21 includes a lower flange 22 having a generally flat bottom surface 23, an annular side surface 24 which projects upwardly from bottom surface 23 and is generally parallel to side wall 15. A cylindrical part 25 of septum 21 projects upwardly from lower flange 22 and terminates in a rounded head 26. Head 26 projects vertically upwardly beyond the upper surface of upper wall 13. As shown in FIGS. 3 and 4, the bottom surface 23 of flange 21 defines the uppermost extent of reservoir 16. The septum 21 in the illustrated embodiment is mechanically interlocked with housing part 11 through the engagement of flange 22 within recess 28, and if desirable or necessary, septum 21 can be further secured to housing part 11 with adhesive.

The lower cage-like member 12 includes a pair of laterally-spaced and generally parallel front and rear walls 30 and 31. Walls 30 and 31 have respective inner surfaces 32 and 33, and respective outer surfaces 34 and 35. Each front and rear wall 30 and 31 has a frusto-conical shape as shown in FIGS. 1 and 4, with the base of the cone located adjacent housing part 11. A plurality of openings 36, and here six, are defined within each of the front and rear walls 30 and 31 and extend between the respective inner and outer surfaces thereof. In the illustrated embodiment, the openings 36 are arranged in two horizontal rows of three openings 36 and three vertical columns of two openings 36 across the lateral extent of the respective front and rear walls 30 and 31. A pair of end walls 37 extend transversely between adjacent pairs of edges of the front and rear walls 30 and 31 at opposite ends of the implant 10. Each end wall 37 has inner and outer oppositely facing surfaces 38 and 39. Further, each end wall 37 defines therein a plurality of vertically-spaced openings 40, and here two, which openings 40 extend between the inner and outer surfaces 38 and 39 thereof. The lower terminal edge portions of the respective front, rear and end walls are joined through a generally flat bottom wall 45. Bottom wall 45 is joined to each of the front and rear walls 30, 31 by a curved edge portion 46, and to each of the end walls 37 by a curved edge portion 47. The bottom wall 45 and its transition into the front, rear and end walls through curved edge portions 46 and 47 forms a solid blunt end of implant 10 which lends rigidity to the lower member 12.

In the illustrated embodiment, the upper housing 11 and the cage-like member 12 are preferably constructed of surgical-grade stainless steel, and the upper housing 11 and cage-like member 12 may be secured to one another with adhesive. However, these components may also be constructed of non-reactive, injection-molded plastic, resin or titanium. The septum 21 in the illustrated embodiment is preferably constructed of silicone or other non-reactive rubber materials.

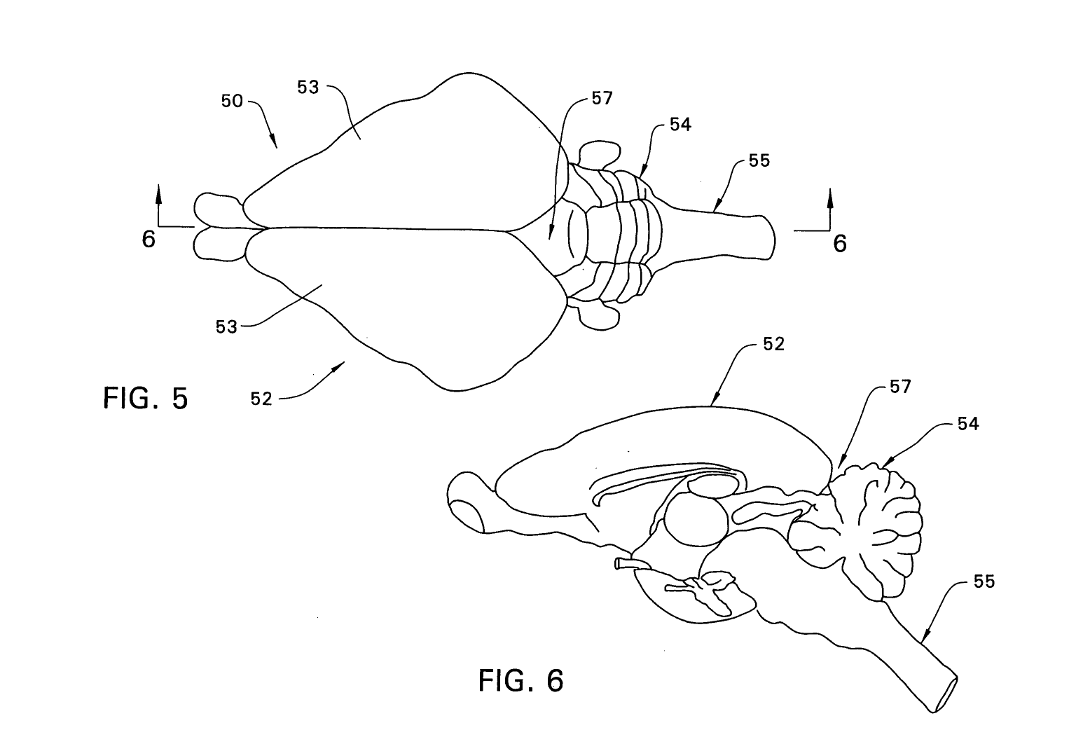

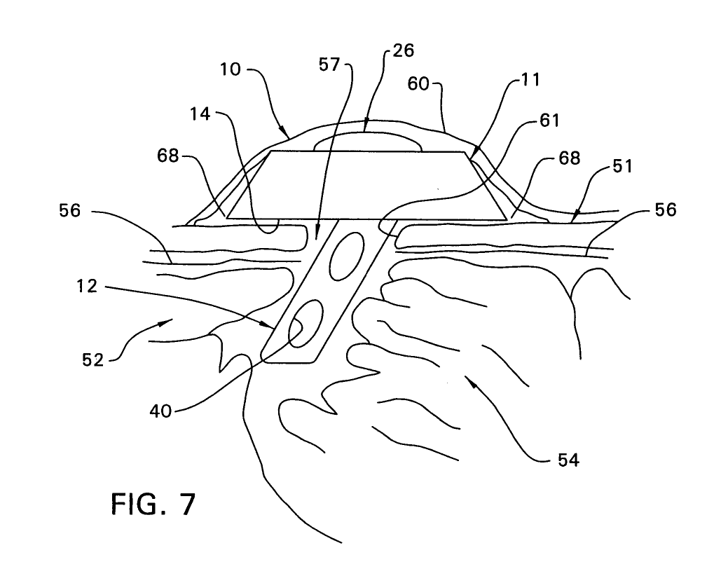

FIGS. 5-7 illustrate a brain 50 of an animal, and particularly the brain of a rat. The brain 50 is contained within the skull 51 (shown only partially in FIG. 7), and includes three primary parts, the cerebrum 52 which is defined by the cerebral hemispheres 53, the cerebellum 54 and the spinal cord or brain stem 55. A thick and fibrous membrane called the dura mater 56 (shown in FIG. 7) lines the interior of the skull 51. As best shown in FIGS. 6 and 7, an area of the brain 50 called the transverse fissure 57 is defined between the cerebrum 52 and the cerebellum 54.

The most common sites for accessing cerebrospinal fluid in the brain are the lateral ventricles (not shown). However, cerebrospinal fluid bathes the entire surface of the brain and tends to pool or collect at various sites within the brain and closer to the skull 51 beneath the dura mater 56, and at least one of these sites is located adjacent to the transverse fissure 57 mentioned above. The implant 10 according to the present invention thus utilizes the transverse fissure 57 to access cerebrospinal fluid as discussed below.

The device 10 according to the invention is implanted within the brain 50 of the animal as follows, with reference to FIG. 7. The skin 60 is incised along the top of the head, and is retracted and musculature is stripped away from the midline of the skull. In rats, there is a landmark having the shape of an inverted “Y” on the top of the head, and the back end of the “Y” is drilled to form a pilot hole through the skull until the dura mater 56 is reached. An operating microscope is utilized at this juncture, and the hole is adjusted as necessary so that the space between the cerebellum 54 and cerebrum 52 can be visualized. This hole is then extended in both the longitudinal and transverse directions through the skull 51 so as to define an opening 61 which is large enough to allow passage of the lower cage-like member 12 into the transverse fissure 57 defined between the cerebellum 54 and cerebrum 52. The dura mater 56 is then incised to allow passage of the cage-like member 12 with the position being modified as needed to avoid major blood vessels. A thin bead of surgical-grade cyanoacrylate gel is placed around the rim of the opening 61 and the implant 10 is pushed into place between the cerebellum 54 and cerebrum 52 and pushed down into the gel until the lower wall 14 of the housing part 11 rests against the outer surface of the skull 51. An adhesive 68, such as dental acrylic, is utilized to build up the skull 51 around the housing part 11 to lock the implant 10 in place on the skull 51. The skin 60 is then sutured so that same completely covers the housing part 11 and the septum 20. Once the implant 10 is in place, the lower cage-like member 12 serves to hold the cerebrum 52 and the cerebellum 54 apart.

Since cerebrospinal fluid is under pressure in the brain 50, this fluid will flow through the openings 36 and 40 defined in the lower cage-like member 12 and into the reservoir 16 of upper housing part 11. This fluid has it's own currents which serve to keep cerebrospinal fluid continuously flowing in and out of the reservoir 16, thereby preventing stagnation of the fluid. When sampling of cerebrospinal fluid is desirable or necessary, the skull 51 of the animal is felt with the fingers in order to locate the bump or nodule created by the upper housing part 11 and septum 21 of the implant 10. Using standard aseptic practices (all personnel wearing surgical masks and bonnets, the use of sterile surgical gloves, sterile supplies and infusates, and a surgical style skin preparation involving 3-5 alternating scrubs with povidone iodine or chlorhexidene soaps followed by 70% isopropyl alcohol and a final application of povidone iodine solution or film), a collection needle is then pushed through the skin 60, through the septum 21 and into the reservoir 16, and cerebrospinal fluid is withdrawn therefrom into the needle. New cerebrospinal fluid will then replace that which was removed from the reservoir 16. Further, the structure of the lower member 12 and the location of the openings provided in the front, rear and end walls prevent normal brain pressure and movement from plugging all of the openings, such that regardless of how the brain shifts or pushes on lower member 12, all of the openings cannot be plugged at one time so cerebrospinal fluid will always be present in the reservoir 16. The same procedure is utilized when dosing of a drug or drugs is desirable or necessary, except that a dosing needle is utilized instead of a collection needle and serves to deliver a drug or drugs directly into the cerebrospinal fluid via the reservoir 16.

While the implant 10 according to the invention is described herein for use with rats, it will be appreciated that the device can be utilized with other animals, such as rabbits, canines, etc. In this regard, the angle a defined between the front wall 30 of the lower cage-like member 12 and the lower wall 14 of the housing part 11 in the illustrated embodiment is approximately 60 degrees based upon the brain structure of a rat. It will be understood that this angle a is chosen based upon the particular brain structure of the animal in which the implant 10 is to be utilized, and it is contemplated that the angle a would range between about 60 degrees and about 75 degrees for various small animals. It is also contemplated that the implant 10 according to the invention can be used in humans.

The implant 10 is ideal for use on small animals, particularly since the lateral ventricles and cisterna magna which are typically utilized to sample or dose cerebrospinal fluid are quite small, and are thus extremely difficult to access via conventional devices. The implant 10 instead creates a port through which cerebrospinal fluid, which tends to pool in areas under the dura mater of the brain, can be repeatedly accessed in a conscious animal without the need for anesthesia, and creates less trauma and discomfort to the animal than conventional methods.

As discussed above, the implant 10 according to the invention can be utilized with different animals, and also humans, and thus the size thereof will be based upon the brain size and structure of the particular animal. With respect to the illustrated embodiment in which the implant 10 is utilized with a rat, the lower cage-like member 12 has a minimum or lower length L1 (FIG. 4) which equals approximately 3 mm, an upper or maximum length L2 of approximately 4 mm, and a height H (FIG. 1) of approximately 4 mm. The width L3 (FIG. 3) of lower cage-like member 12 is approximately 1.5 mm.

It will be appreciated that the number of and the pattern of openings 36 and 40 defined in lower member 12 illustrated herein are only one example of a particular configuration of lower member 12, and other patterns and numbers of such openings are within the scope of the invention.

Although a particular preferred embodiment of the invention has been disclosed in detail for illustrative purposes, it will be recognized that variations or modifications of the disclosed apparatus, including the rearrangement of parts, lie within the scope of the present invention.

1. An implant for accessing cerebrospinal fluid in the brain which includes a cerebrum and a cerebellum enclosed by a skull, said implant comprising an upper housing part for positioning on the skull, said housing part including a septum and defining a reservoir therein adjacent said septum, and a rigid lower part cantilevered downwardly from said housing part for positioning in an area of the brain defined between the cerebrum and the cerebellum, said lower part defining an opening therein which communicates with said reservoir and the area defined between the cerebrum and the cerebellum to permit access to cerebrospinal fluid located adjacent the area between the cerebrum and the cerebellum.

2. The implant of claim 1 wherein said lower part is rigidly secured to said housing part at a predetermined angle relative to said housing part based upon the structure of the brain.

3. The implant of claim 1 wherein said septum and said housing part together define said reservoir.

4. The implant of claim 1 wherein said lower part comprises a cage-like structure which defines a hollow interior in communication with said reservoir and a plurality of said openings therein which communicate with said hollow interior and permit the flow of cerebrospinal fluid through said cage-like structure.

5. The implant of claim 4 wherein said cage-like structure is defined by spaced-apart front and rear walls respectively facing the front and rear of the brain, a pair of spaced-apart end walls extending transversely between and interconnecting said front and rear walls, and a bottom wall extending between lower edge portions of said front, rear and end walls, some of said walls defining openings therein.

6. The implant of claim 5 wherein said openings are defined in said front, rear and end walls.

7. A surgical implant positioned subcutaneously on the skull of a small animal for providing access to cerebrospinal fluid in the brain of the animal, said implant comprising an upper portion having a lower surface positioned on the skull and a septum mounted within an upwardly opening recess defined in said upper portion, said upper portion defining therein a chamber for storing cerebrospinal fluid adjacent said septum, and a lower portion which projects into an area of the brain between the cerebrum and the cerebellum in which cerebrospinal fluid pools, said lower portion defining a plurality of openings therein which communicate with said chamber.

8. The surgical implant of claim 7 wherein said upper and lower portions are constructed of a rigid material and said lower portion is oriented at a predetermined angle relative to said lower surface based upon the brain structure of the small animal.

9. The surgical implant of claim 7 wherein said lower portion is constructed of a material having a rigidity sufficient to push the cerebrum and the cerebellum apart to permit flow of cerebrospinal fluid into said reservoir.

10. The surgical implant of claim 7 wherein said septum defines a portion of said chamber and said septum is penetrable to permit collection of cerebrospinal fluid from said chamber and to permit dosing of therapeutic agents into the cerebrospinal fluid.

11. The surgical implant of claim 7 wherein said septum is penetrable to permit at least one of collection of cerebrospinal fluid from said chamber and administration of therapeutic agents into the cerebrospinal fluid in the brain.

12. The surgical implant of claim 8 wherein said upper portion has a disc-like shape and said lower portion is cantilevered downwardly from said lower surface of said upper portion and extends along a significant part of the diameter of said upper portion.

13. A method of surgically inserting an implant into the brain of a patient, said method comprising the steps of:
providing an implant having an upper housing part having a septum and defining a chamber therein adjacent the septum, and a lower part cantilevered downwardly from the upper housing part and defining at least one opening therein in communication with the chamber;
locating an area of the brain defined between the cerebrum and the cerebellum containing cerebrospinal fluid;
making an opening in the skull at said area;
inserting the lower part into said area until the upper part is positioned on the skull.

14. The method of claim 13 including positioning the upper part subcutaneously.

15. The method of claim 13 wherein said step of inserting includes pushing the cerebrum and the cerebellum apart with the lower part.

16. The method of claim 13 including surgically inserting the implant in the brain of a small animal, such as a rat.

17. A method of accessing cerebrospinal fluid from the brain, said method comprising the steps of:
providing an implant having a upper part having a septum and defining therein a reservoir adjacent said septum, and a lower part cantilevered downwardly from said upper part and defining an opening therein which communicates with said reservoir;
embedding the implant within the brain so that the lower part projects into the transverse fissure of the brain between the cerebrum and the cerebellum and the upper part is positioned subcutaneously on the skull; and
accessing cerebrospinal fluid within said reservoir through said septum.

18. The method of claim 17 including inserting a collection needle through said septum and into said reservoir and collecting cerebrospinal fluid.

19. The method of claim 17 including inserting a dosing needle through said septum and into said reservoir and dosing the cerebrospinal fluid with a therapeutic agent.

20. The method of claim 17 including holding the cerebrum and the cerebellum apart from one another with the lower part.
